(19) United States
(12) Patent Application Publication
(10) Pub. No.: US 2026/0355316 A1
(43) Pub. Date: Nov. 12, 2026

(54) Sipuleucel-T & N-803

(71) Applicant: Nant Holdings IP, LLC, Culver City, CA (US)

(72) Inventor: Patrick Soon-Shiong, Culver City, CA (US)

(73) Assignee: Nant Holdings IP, LLC, Culver City, CA (US)

(21) Appl. No.: 19/669,252

(22) Filed: May 6, 2026

Related U.S. Application Data
(60) Provisional application No. 63/802,005, filed on May 8, 2025.

(51) Int. Cl. A61K 40/24; A61K 35/15; A61K 35/17; A61K 38/17; A61K 38/19; A61K 38/20; A61K 38/46; A61K 40/11; A61K 40/15; A61K 40/17; A61K 40/42; A61P 35/04

(52) U.S. Cl. CPC A61K 40/24; A61K 35/15; A61K 35/17; A61K 38/1793; A61K 38/193; A61K 38/2086; A61K 38/465; A61K 40/11; A61K 40/15; A61K 40/17; A61K 40/42; A61P 35/04; C12Y 301/03002

(57) ABSTRACT

Compositions and methods for treatment of mCRPC are presented in which apheresis-derived peripheral blood mononuclear cells (PBMCs) are activated with a tumor antigen and granulocyte-macrophage colony stimulating factor (GM-CSF). Activation is enhanced in vitro by exposure of the PBMCs with IL15:IL15RαSu or an agonist derivative thereof, and in some embodiments additionally in vivo by administering the IL15:IL15RαSu or agonist derivative prior to and/or after transfusion of the activated PBMCs.

This application claims priority to our copending US Provisional patent application with the Ser. No. 63/802,005, filed May 8, 2025, incorporated by reference herein.

### FIELD OF THE INVENTION

The field of the invention is compositions and methods of using nogapendekin alpha imbakicept (N-803) or derivative thereof in combination with apheresis-derived peripheral blood mononuclear cells (PBMCs) and one or more protein antigens, particularly.

### BACKGROUND OF THE INVENTION

The background description includes information that may be useful in understanding the present invention. It is not an admission that any of the information provided herein is prior art or relevant to the presently claimed invention, or that any publication specifically or implicitly referenced is prior art.

All publications and patent applications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Prostate cancer is the most common malignancy affecting men. Between 2003-2017, 3.1 million new cases of prostate cancer were recorded. Seventy-seven percent of these cases were localized while 5% had distant metastases. Based on the American Cancer Society 2023 Cancer Statistics, prostate cancer was the second most common cause of death in men behind lung cancer. While localized prostate cancer is relatively more favorable, metastatic prostate cancer carries a grim prognosis. The 10-year overall survival for localized prostate cancer is close to 100% while the 10-year survival for distant metastatic prostate cancer was ~18.5% based on prior SEER analysis.

The most advanced form of the cancer is metastatic castrate resistant prostate cancer (mCRPC) which is challenging to treat, and prior to 2004, there was a scarcity of feasible options.

In the last 2 decades, several agents have been approved that provide modest survival benefit to men of several months on average. Prostate cancer-related mortality has been declining, likely due to the expanded treatment options and earlier detection by screening methods. Five-year relative survival improved from 28.7% in 2001-2005 to 32.3% between 2011-2016 for metastatic prostate cancer. The prevalence of the disease and limited improvements in outcomes for newly approved therapies underlines the critical need for more effective therapeutic options.

Sipuleucel-T (Sip-T) is the only FDA approved cellular therapeutic vaccine for mCRPC patients and the only immunotherapy approved for the disease. It was FDA-approved in 2010 and remains as a standard of care and NCCN guideline recommended treatment for asymptomatic to minimally symptomatic mCRPC patients. Sipuleucel-T is most effective in patients with lower tumor burden, such as a Gleason score of 7 or less. However, activation, tumor-infiltration, and tumor suppression are often less than desirable, with a reported median survival benefit of 4.1 months. The median overall survival in a clinical study for Sip-T was shown to be 25.8 months in the Sip-T group compared to 21.7 months in the placebo. The median time to disease progression was similar in both groups, however.

In an effort to increase therapeutic efficacy, Sip-T has been studied in conjunction with IL7 (see J Immunother Cancer 2021 August; 9 (8):e002903), where eligible mCRPC patients were randomized to observation or IL7 after finishing Sip-T. The group treated with IL7 had a significant increase in peripheral CD4, CD8, and NK cells. While there were no significant increases in PA2024 specific IFN gamma response between the groups, the IL7 arm showed significant increases in PAP-specific T cell proliferation and IgG responses over time, whereas none were seen in the observation arm. PSA doubling times of greater than 6 months (a surrogate for disease kinetics) were seen to be twice as common in the IL7 group compared to the observation group. The combination of Sip-T and IL7 was also found to be safe and tolerable, with only 7.1% of patients in the IL7 group having any grade 3+ treatment-related adverse events (TRAE). However, the clinical development of the recombinant IL7 used (CYT107) was halted due to efficacy shortfalls, safety concerns, and adverse events.

In another approach, Sip-T was studied in conjunction with ipilimumab after Sip-T (Immunotargets Ther. 2017 Mar. 20; 6:11-16). Here, patients received ipilimumab either immediately after finishing Sip-T or 3 weeks later. Antigen specific B and T cell responses were evaluated and the timing of ipilimumab did not seem to affect the immune responses. Here, patients received ipilimumab either immediately after finishing Sip-T or 3 weeks later, and antigen specific B and T cell responses were evaluated. Unfortunately, the timing of ipilimumab did not seem to affect the immune responses. In a subsequent investigation, co-culture of Sip-T with IL15 and control or prostate-relevant antigens showed a substantial activation and expansion of CD8+ T cells and NKT cells in vitro in an antigen-specific manner, and adoptive transfer of IL 15-treated sip-T into NSG mice resulted in more potent prostate tumor growth inhibition compared with control sip-T (see Cancer Immunol Res. 2024 May 2; 12(5):559-574). Here, evaluation of tumor-infiltrating lymphocytes revealed a 2- to 14-fold higher influx of sip-T and a significant increase in IFNγ producing CD8+ T cells and NKT cells within the tumor microenvironment in the IL15 group. However, the efficacy of such treatment in humans was not reported.

N-803 is a chimeric IL-15 super-agonist with extended half-life as compared to IL-15 and has been studied in human subjects at doses of both 10 ug/kg and 20 ug/kg. Here, the half-life was about 20 hours and N-803 was generally well tolerated. N-803 has also been studied in early phase clinical trials in combination with other drugs. For example, N-803 was studied with rituximab in relapsed/refractory indolent non-Hodgkin lymphoma in a Phase 1 dose escalation trial, and the study showed increased NK cells and T cells at the end of the trial. In another combination, N-803 was evaluated with BCG for non-muscle invasive bladder cancer. Here, patients with BCG-unresponsive NMIBC received a combination of BCG and N-803 and there was a 99% bladder cancer specific overall survival at 2 years.

Thus, even though various compositions and methods of prostate cancer treatments are known in the art, all or almost all of them suffer from several drawbacks. Therefore, there remains a need for improved compositions and methods for prostate cancer treatment, particularly where a patient is diagnosed with mCRPC, where the treatment is well-tolerated, and where antigen activation, tumor-infiltration and/or tumor suppression are improved.

### SUMMARY OF THE INVENTION

The inventive subject matter is directed to various compositions and methods of improved therapy of a subject diagnosed with prostate cancer, and especially mCRPC, in which apheresis-derived peripheral blood mononuclear cells (PBMCs) are activated with an antigen, and especially a protein antigen comprising a fusion proteins of a prostatic acid phosphatase (PAP) and a granulocyte-macrophage colony stimulating factor (GM-CSF). Moreover, activation is further enhanced by exposure of the PBMCs with IL-15, IL15:IL15RαSu, or an agonist derivative thereof to so increase activity of antigen presenting cells, T cells, and NK cells and to thereby increase tumor-infiltration and/or tumor suppression. In further contemplated aspects of the compositions and methods presented herein, the subject has lymph node involvement and/or distal tumors.

In one aspect of the inventive subject matter, the inventor contemplates a pharmaceutical composition that comprises apheresis-derived peripheral blood mononuclear cells (PBMCs), IL-15, IL15:IL15RαSu, or an agonist derivative thereof, and a protein antigen. In most embodiments, the protein antigen is a fusion protein, and especially contemplated fusion proteins include a prostatic acid phosphatase (PAP) and a granulocyte-macrophage colony stimulating factor (GM-CSF). Thus, and among other examples, a suitable fusion protein is PA2024. Similarly, where an agonist derivative of IL15:IL15RαSu is used, it is especially contemplated that the derivative is nogapendekin alpha inbakicept (N-803).

With respect to suitable cells, it is typically contemplated that the PBMCs are from a human subject (typically diagnosed with prostate cancer), and/or that the PBMCs are enriched for monocytes, T cells, or natural killer (NK) cells. Therefore, in at least some embodiments, the PBMC are treated (typically ex vivo) with N-803 and PA2024, thereby stimulating the loading and MHC presentation of PAP by antigen presenting cells.

Viewed from a different perspective, the inventor therefore also contemplates a pharmaceutical composition comprising 1) IL-15, IL15:IL15RαSu, or an agonist derivative thereof and 2) sipuleucel-T (Sip-T). As noted before, the agonist derivative of IL15:IL15RαSu is preferably, but not necessarily, nogapendekin alpha inbakicept (N-803). Moreover, the inventor also contemplates a method of treating a patient having metastatic castration-resistant prostate cancer (mCRPC) that includes a step of obtaining peripheral blood mononuclear cells (PBMCs) from the patient, and a further step of activating ex vivo the PBMCs with N-803 and a PA2024 fusion protein containing prostatic acid phosphatase to provide an enhanced immunotherapy sipuleucel-T (Sip-T). In another step, the enhanced immunotherapy Sip-T is then administered to the patient.

Similarly, the inventor contemplates a method of treating a patient having metastatic castration-resistant prostate cancer (mCRPC) where the method includes a step of obtaining peripheral blood mononuclear cells (PBMCs) from the patient, and a further step of activating ex vivo the PBMCs with N-803 and PA2024 fusion protein containing prostatic acid phosphatase to provide an enhanced immunotherapy sipuleucel-T. In yet another step, N-803 and the enhanced immunotherapy sipuleucel-T is administered to the patient. Likewise, the inventor contemplates a method of treating a patient having metastatic castration-resistant prostate cancer (mCRPC) where the method includes a step of obtaining peripheral blood mononuclear cells (PBMCs) from the patient, and a further step of activating ex vivo the PBMCs with PA2024 fusion protein containing prostatic acid phosphatase to provide an immunotherapy sipuleucel-T. In yet another step, N-803 and the immunotherapy sipuleucel-T are administered to the patient.

Therefore, and viewed from yet another perspective, the inventor also contemplates a method of stimulating CD8+ T cells in a patient having metastatic castration-resistant prostate cancer (mCRPC). Such method will typically include the steps of obtaining peripheral blood mononuclear cells (PBMCs) from the patient, activating ex vivo the PBMCs with N-803 and PA2024 fusion protein containing prostatic acid phosphatase to provide an enhanced immunotherapy sipuleucel-T, and administering the enhanced immunotherapy sipuleucel-T to the patient.

Additionally, the inventor also contemplates a method of stimulating CD8+ T cells in a patient having metastatic castration-resistant prostate cancer (mCRPC), where the method includes the steps of obtaining peripheral blood mononuclear cells (PBMCs) from the patient, activating ex vivo the PBMCs with N-803 and PA2024 fusion protein containing prostatic acid phosphatase to provide an enhanced immunotherapy sipuleucel-T, and administering N-803 and the enhanced immunotherapy sipuleucel-T to the patient.

Similarly, the inventor contemplates a method of stimulating CD8+ T cells in a patient having metastatic castration-resistant prostate cancer (mCRPC) where the method includes the steps of obtaining peripheral blood mononuclear cells (PBMCs) from the patient, activating ex vivo the PBMCs with PA2024 fusion protein containing prostatic acid phosphatase to provide an immunotherapy sipuleucel-T, and (typically contemporaneously) administering N-803 and the immunotherapy sipuleucel-T to the patient.

In view of the above, it should therefore also be appreciated that the inventor contemplates a kit for use in the immunostimulation of apheresis-derived PBMCs. Most typically, the kit will comprise 1) IL15, IL15:IL 15RαSu, or an agonist derivative thereof, and 2) PA2024, and directions for use thereof. Alternatively, contemplated kits for use in the preparation of enhanced Sipuleucel-T for use in the treatment of castration-resistant prostate cancer may also comprise a first composition comprising IL15:IL15RαSu or an agonist derivative thereof, PA2024, and directions for use in conjunction with prepared patient-derived PBMCs; and a second composition comprising IL15:IL15RαSu or an agonist derivative thereof and the first composition; wherein the therapeutic product of the first composition is for administration with the IL15:IL15RαSu or an agonist derivative thereof of the second composition.

In still further embodiments, the inventor also contemplates a method of stimulating antigen presenting cells to present antigenic peptides via cell surface expressed major histocompatibility complex proteins. In such method apheresis-derived peripheral blood mononuclear cells (PBMCs), IL15:IL15RαSu or an agonist derivative thereof, and a protein antigen are combined ex vivo. In at least some embodiments, the protein antigen is a fusion protein (e.g., comprising a prostatic acid phosphatase (PAP) and granulocyte-macrophage colony stimulating factor (GM-CSF), such as PA2024). Moreover, it is contemplated that the agonist derivative of IL 15: IL 15RαSu is nogapendekin alpha inbakicept (N-803), that the PBMCs are from a human subject, and/or that the PBMCs are enriched for monocytes, T cells, or natural killer (NK) cells. For example, the PBMC may be treated with N-803 and PA2024, thereby stimulating the loading and MHC presentation of PAP by antigen presenting cells.

Alternatively, the inventor further contemplates a method of enriching antigen presenting cells for cell surface expression of major histocompatibility complex protein I (MHC-I). Such method will typically comprise a step of ex vivo combining apheresis-derived peripheral blood mononuclear cells (PBMCs), IL15:IL15RαSu or an agonist derivative thereof, IL12 or an agonist derivative thereof, IL-18 or an agonist derivative thereof, and a protein antigen. For example, in some embodiments the PBMC may be enriched for NK cells, and the NK cells may comprise or be enriched in memory-like cytokine enhanced NK cells (m-ceNK). Alternatively, or additionally, the NK cells will be (enriched in) cytotoxic for MHC-I negative APC. In such example, it is generally preferred that the APC are enriched for MHC-I expression.

Various objects, features, aspects and advantages of the inventive subject matter will become more apparent from the following detailed description of preferred embodiments.

### BRIEF DESCRIPTION OF THE DRAWING

FIG. 1 is an exemplary flow chart of a study for treatment using a protocol according to the inventive subject matter.

FIG. 2 is an exemplary illustration of the cohort treatments of FIG. 1.

FIG. 3 is an exemplary illustration of administration schedule of Sip-T and N-803.

### DETAILED DESCRIPTION

The inventor has discovered various compositions and methods of treatment of prostate cancer, and especially mCRPC (which may or may not have spread to the lymph nodes and/or other distal sites), in which sipuleucel-T treatment is augmented with N-803. In some embodiments, augmentation may be ex vivo during activation of the apheresis product, in other embodiments augmentation may be in vivo before and/or after infusion of the activated apheresis product, and still further embodiments, augmentation is ex vivo during activation of the apheresis product and in vivo before and/or after infusion of the activated apheresis product.

For example, one contemplated exemplary method of treatment includes an apheresis step in which peripheral blood mononuclear cells (PBMCs) are obtained from a subject, which will typically be the subject previously diagnosed with metastatic castration-resistant prostate cancer (mCRPC). Upon preparation of the PBMC fraction, the PBMCs are then exposed to PA2024, a fusion protein comprising prostatic acid phosphatase (PAP) and granulocyte-macrophage colony stimulating factor (GM-CSF) in the presence of nogapendekin alpha inbakicept (N-803) following a protocol as known for the preparation of sip-T. However, in this protocol, IL-15 is replaced by N-803 at an equivalent concentration (typically 50 ng/ml). Upon conclusion of activation, the cells are then washed with transfusion media (e.g., isotonic ringer solution) and administered once every two weeks to the subject at a dosage of at least 5×10⁷ CD54⁺ cells over three transfusions.

In another example, the inventor contemplates the same method as noted immediately above, however in this example, the transfusion protocol is preceded by a single subcutaneous injection of N-803 at a dosage of between 5-20 mcg/kg one week prior to the transfusion. Alternatively, or additionally, N-803 can also be administered via single subcutaneous injection at a dosage of between 5-20 mcg/kg one week after transfusion. Alternatively, an adenoviral vector comprising a nucleic acid sequence encoding N-803 may be administered intravenously.

As will be readily appreciated, while N-803 is generally preferred in the compositions and methods contemplated herein, various alternative cytokines are also deemed suitable, and especially contemplated cytokines include IL-7, IL-12, IL-15, IL-18, IL-21, and any reasonable combination and fusion proteins thereof. Moreover, it is generally preferred that N-803 is used for activation and/or leading/trailing administration. However, in at least some aspects, IL15:IL15RαSu or other agonist derivative thereof are also expressly contemplated. The inventors contemplate IL-15 and IL-15Ra derivatives as fusion constructs, wherein in vitro and in vivo stability are enhanced by fusion to an Ig Fc domain, Tissue Factor, or any protein which confers stability on the construct. The fusion constructs as conceived are all functional IL-15 based agonists at IL-15Rβγ. IL-15 derivatives also comprise mutant IL-15 proteins which have increased (superagonist) or decreased potency at the IL-15Rβγ. An especially favored superagonist mutation is N72D, wherein the numbering is with respect to the sequence of wild type human IL-15.

While not wishing to be bound by any theory or hypothesis, the inventor contemplates that N-803 may have superior activation effect in the PBMC fraction relative to IL-15 due to the presence of the Fc portion in the N-803 (or other fusion molecule). Such Fc portion may interact with various Fc receptors on immune cells such as CD16, CD32, and/or CD64 that can be found, among others on NK cells and NKT cells. As such, N-803 may be particularly beneficial in activation of a PBMC fraction and immune cells therein. Still further, and particularly where the treatment method includes a leading and/or trailing administration of N-803 (i.e., administration of N-803 prior and/or after transfusion of sip-T), use of N-803 may beneficially enhance/activate immune cells prior to apheresis and/or enhance/activate immune cells post administration of sip-T. Such enhancement/activation is deemed to increase antigen processing in vivo, as well as tumor-infiltration and/or tumor suppression. Additionally, leading and/or trailing administration of N-803 may also increase the cell count on NK cells, NKT cells, and/or T cells. Where leading administration of N-803 is present, apheresis may therefore also increase a fraction of activated/activatable immune cells.

In still further contemplated aspects, it should be appreciated that while the antigen for treatment of mCRPC is preferably PA2024, other forms of fusion proteins between prostatic acid phosphatase (PAP) and a granulocyte-macrophage colony stimulating factor (GM-CSF) are also deemed suitable for use herein. Similarly, the antigen need not be a fusion protein, but may also be a single prostate cancer specific antigen, alone or in combination with GM-CSF. Additionally, or alternatively, the prostate cancer specific antigen may also be a patient and tumor specific (neo) antigen. Where a cancer other than prostate cancer is treated, it should be noted that the antigen presented to the PBMCs may be any cancer-specific antigen (e.g., MUC-1, CEA, brachyury, etc.), tumor specific (e.g., CDK4, Her2, BCR-ABL, MART-2, etc.), or patient and tumor specific neoantigens identified from omics analysis of a tumor versus matched normal tissue. Such antigens may be prepared as fusion proteins (with or without linker) or together with GM-CSF.

Suitable dosages for leading and/or trailing N-803 injections will typically be between 0.1 and 25 mcg/kg, preferably via subcutaneous injection, at least once weekly, or at least once monthly, bimonthly, and more. However, in particularly contemplated embodiments, N-803 is administered at a dosage between 5 mcg/kg and 25 mcg/kg, once weekly. Suitable dosages for sip-T as prepared herein will be between 1×10⁶ and 1×10⁹ cells/transfusion, and more typically between about 1×10⁷ and 1×10⁸/transfusion (e.g., at least 5×10⁷ cells/transfusion) with most typically between and and five transfusions separated by one to two weeks. For example, a typical schedule of administration may be three transfusions of 5×10⁷ cells/transfusion over a period of three weeks. Where multiple transfusions are preferred, it is contemplated that the sip-T as presented herein may be freshly prepared, or drawn form a cryopreserved quantity.

### EXAMPLES

The following provides exemplary guidance for evaluation, quantitation, and/or confirmation of aspects of the inventive subject matter presented herein. However, this guidance is not intended to limit the scope of the invention.

### Preliminary Study:

Under an IRB approved protocol, the inventor collected sip-T product from patients for detailed analysis by mass cytometry (CyTOF). Both recombinant IL-15 and N-803 were used to stimulate sip-T in vitro. The inventor then established a preclinical mouse model of human prostate cancer to examine the impact of IL-15 or N-803 on sip-T efficacy. CyTOF, flow cytometry, RNAseq, and qPCR were used to evaluate the phenotype, function, and in vitro/in vivo changes seen in combination therapy.

CyTOF analysis (data not shown) revealed that CD3⁺ T cells constituted the highest proportion of sip-T, followed by B-cells, natural killer (NK) cells, NKT, and monocytes/dendritic cells. Both IL15 and N-803 significantly expanded and activated of CD8+ T-cell and NK cell populations. Co-culture of sip-T with IL-15 and control or prostate-relevant antigens showed significant activation and expansion of CD8 T and NKT cells in an antigen-specific manner, with significantly increased tumor cytotoxicity and evidence of antigen spread. IL-15 or N-803 treatment combined with sip-T showed significant suppression of tumor growth in vivo compared to sip-T alone, with increased tumor-infiltrating lymphocytes (TIL). A significant increase in interferon-γ producing CD8+ T and NKT TIL was seen in the IL-15 and N-803 groups. Tumor transcriptomic analyses revealed IL-15 treatment was able to reverse immunoresistance. Both IL-15 and N-803 treatment significantly enhanced anti-tumor efficacy, effector immune cell activation, and tumor TIL infiltration. These studies support the ongoing development of a clinical trial with N-803 and sip-T in mCRPC patients.

In one embodiment, the inventor contemplates an exemplary contemplated course of treatment that includes three basic steps: (1) The patient's white blood cells, primarily dendritic cells, a type of antigen-presenting cells (APCs), are extracted in a leukapheresis procedure. (2) The blood product is sent to a production facility and incubated with a fusion protein (PA2024, comprising the antigen prostatic acid phosphatase (PAP), which is present in 95% of prostate cancer cells and an immune signaling factor granulocyte-macrophage colony stimulating factor (GM-CSF) that helps the APCs to mature); and (3) The activated blood product (APC8015) is returned from the production facility to an infusion center and reinfused into the patient. Such treatment may be complemented with a leading and/or trailing N-803 administration, typically one week prior/after transfusion as described above.

### Human Study:

Changes in tumor and peripheral immune responses over time will be assessed. Blood and tissue will be collected during the study and analysis planned at a later date on frozen stored blood and tissue samples. Noninvasive PET imaging will be performed to further investigate tumor and immune responses. It is contemplated that upon conclusion of treatments according to the inventive subject matter blood markers, tissue samples, and imaging will reveal a significant improvement, including extension of failure free survival, progression free survival, overall survival, reduction of PSA/PSMA, reduction of metastatic burden, reduction of tumor volume and/or number, etc.

PET Imaging: Noninvasive imaging methods can provide detailed information of disease distribution. Functional and molecular imaging with appropriately selected positron emitting radiotracers can provide further data to detect and characterize metabolism and target protein expression. PET imaging will be performed at baseline and post-treatment; scans will use [18F]-Fluorodeoxyglucose ([18F]-FDG) and [68Ga]-PSMA-11, both FDA approved and already widely used in prostate cancer patients. [18F]-FDG is the most widely used PET radiotracer. The uptake and phosphorylation of the molecule reflects glycolytic processes in cells throughout the body and is most commonly used as a means to detect aberrant metabolic activity that reflects the Warburg effect in cancer cells. However, upregulated glucose utilization is also a hallmark of immune cell activity- and common findings of [18F]-FDG avid tissues (for example, lymph nodes following vaccination or infection) that are not cancerous are a confounder, but potentially useful to monitor inflammation. Prostate specific membrane antigen (PSMA) is highly expressed on the majority of metastatic prostate cancers, and specific PET tracers are now in use in a variety of clinical settings in men with prostate cancer. The 68Ga-PSMA-11 tracer has been FDA approved since 12/2020. Use of [18F]-FDG to phenotype or to monitor immunotherapy responses may provide insights into clinical responses when combined with tumor-cell targeted PSMA-11 gallium tracer.

Patients will undergo pre- and post-treatment [18F]-FDG and [68Ga]-PSMA-11 PET scans with low dose correlative CT scans. SUVmax and SUVmean values for the pre- and post-treatment scans for index lesions (defined by CT) will be compared by Wilcoxon signed rank test and correlated to tissue sample measurements (below).

FACS/CyTOF Analysis: Peripheral blood mononuclear cells (PBMCs and tumor-infiltrating leukocytes (TIL), if available, will be evaluated for any changes in peripheral leukocyte subsets induced by treatment. Validated lymphoid and myeloid panels for CyTOF analyses are already established by the inventor. CyTOF provides the advantage of being able to test multiple parameters at the same time, thus increasing yield when limited tumor samples are available, as if often the case.

Serum Cytokines: The expression pattern of multiple cytokines and chemokines in peripheral blood will be assessed to analyze the immune system response using Luminex or MSD.

ctDNA: Cancer Personalized Profiling by deep Sequencing (CAPP-Seq) will be utilized for the quantification of ctDNA. The plasma isolated from donor blood will be analyzed for pre-specified mutations-“selectors”-from population-based public databases. Hybridization capture with the selector probe set will be sequenced to a depth of approximately 10⁴ coverage on the tumor tissue, and the identified mutations will be tested at pre-specified time points in the patients' blood. Plasma cfDNA alterations in the androgen receptor (AR)/enhancer and locus, utilizing targeted sequencing as part of a published assay-EnhanceAR-Seq-will be performed. The assay detected 18 out of 23 resistant cases (78%) and outperformed the CTC AR-V7 assay, which was also run on a subset of patients.

Single cell RNAseq: If available from fresh biopsy tissue, single-cell RNAseq may be performed+/−TCR sequencing+/−CITE-seq. TCR profiling will evaluate for diversity and clonality. The TCR analysis will provide information regarding the changes to the T cell repertoire, if any, if the administration of study drugs at various time points during the clinical trial. Cellular Indexing of Transcriptomes and Epitopes by sequencing (CITE-seq), combines multiplexed antibody-based detection of protein markers with transcriptome profiling of numerous single cells in parallel. This method has the advantage of being able to characterize cellular phenotypes than scRNA alone better.

NGS: Tumor tissue will be analyzed by next-generation sequencing (NGS) for both RNA and DNA, focusing on targeted known mutations in prostate cancer DNA and transcriptome.

Multiplexed Tumor Imaging: If adequate tissue is available, highly multiplexed spatial transcriptomics will be utilized. A customized 1000 probe set encompassing relevant immune and prostate cancer markers will be utilized. The data acquired from each probe is then overlaid with representative pseudocolors to create a composite image with both spatial and intensity information for markers that can be used to characterize different cellular components of the tumor tissue (e.g., T-cell subtypes, proliferation markers; macrophages; vessel; B-cells; etc.). Advanced algorithms will then be applied, and clustering performed.

Study Design: Standard dosing of Sip-T will be used. This includes 3 doses typically given every 2 weeks (according to the FDA label). N-803 has been administered using a variety of doses and schedules, but most commonly has been given weekly via subcutaneous route, typically at dosages between 5 and 25 mcg/kg.

Patient Selection: Suitable patients include those with histologically or cytologically confirmed prostate adenocarcinoma, imaging- or biopsy-proven metastatic disease (may have any type or location of metastases (bone, lymph node, visceral)), and prior treatment including orchiectomy or luteinizing hormone-releasing agonist or antagonist treatment with documented testosterone>50 ng/dL and must be eligible for Sipuleucel-T therapy.

The first cohort of patients enrolling (Cohort 1) will receive the standard dose and schedule of sip-T, with N-803 administered concurrently at the assigned dose level for a total of three doses. Cohort 1 patients are all part of the dose-finding portion of the study and will be monitored for DLTs beginning at the first dose of N-803 and ending 2 weeks after the last dose. Depending on DLTs experienced, N-803 may be de-escalated by two dose levels. Sip-T standard dose is >50 million CD54+ cells administered i.v., typically on Day 1, Day 15, and Day 29. N-803 standard dose will be between 5 and 25 mcg/kg administered s.c., on Day 1, Day 15, and Day 29, and Day 7 or Day 36. N-803 will be given no sooner than 30 after conclusion of Sip-T.

Once the recommended dose of N-803 (when given in combination with sip-T) is found (i.e., the dose level with acceptable toxicity (<33% dose limiting toxicity (DLT) rate)), then enrollment will begin to Cohort 2. Patients enrolled to this cohort will receive an additional dose of single agent N-803 at the RP2D one week after completion of the third Sip-T+N-803 combination dose. RP2D is the highest dose of N-803 that has an overall DLT rate of less than 33% in a total of 6 patients (0 or 1 patients experience DLT). The inventor contemplates that this additional boost/maintenance of N-803 will enhance clinical reaction as compared without the additional boost/maintenance of N-803. Table 1 depicts an exemplary dose-de-escalation schedule.

TABLE 1
Dose De-Escalation Schedule

| Dose Level | N-803 | Sipuleucel-T |
|---|---|---|
| Level 1 (Starting Dose) | 20 mcg/kg S.C. | Standard dose |
| Level −1 | 10 mcg/kg S.C. | (>50 million |
| Level −2 | 6 mcg/kg S.C. | CD54+ cells) |

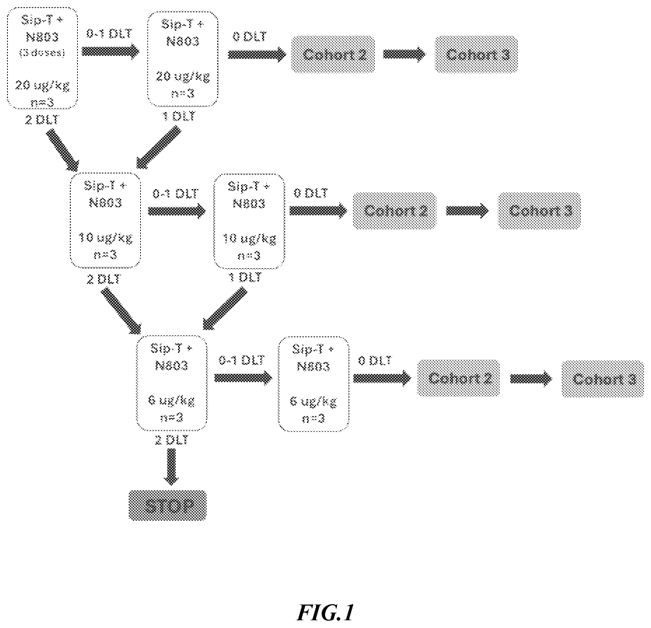

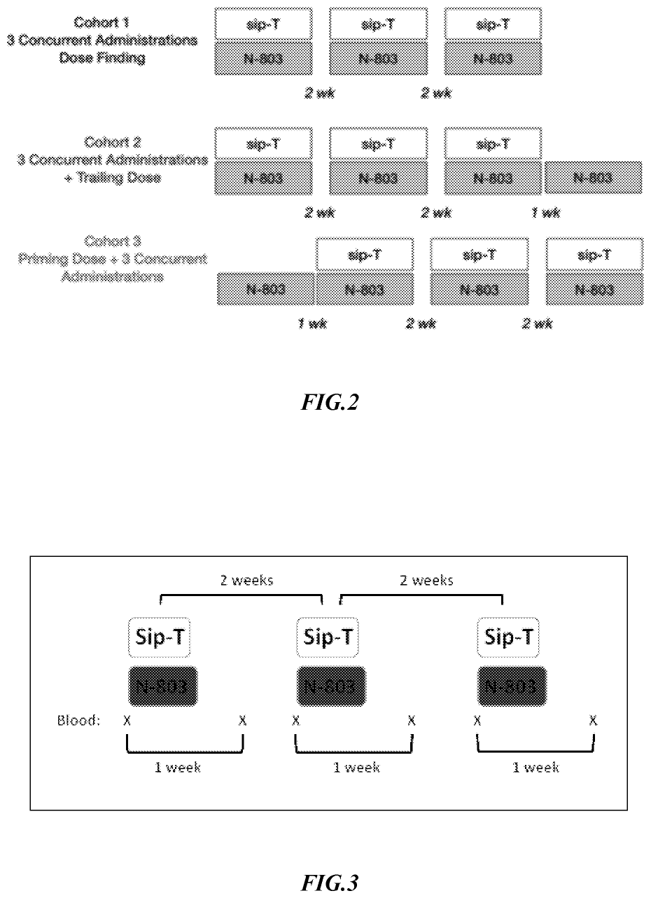

After enrollment has completed to Cohort 2, enrollment will begin to Cohort 3. Patients enrolled to this cohort will receive an additional dose of single agent N-803 at the RP2D one week prior to the first Sip-T+N-803 combination dose. Here as well, the inventor contemplates that this additional boost/maintenance of N-803 will enhance clinical reaction as compared without the additional boost/maintenance of N-803. FIG. 1 is an exemplary flow chart of a study for treatment using a protocol according to the inventive subject matter, and FIG. 2 is an exemplary illustration of the cohort treatment of FIG. 1. FIG. 3 is an exemplary illustration of administration schedule of Sip-T and N-803.

Sipuleucel-T: Sipuleucel-T is an autologous cellular immunotherapy available as a suspension for intravenous infusion. Sipuleucel-T consists of autologous peripheral blood mononuclear cells (PBMC), including antigen presenting cells (APCs) that have been activated during a defined culture period with a recombinant human protein, PAP-GM-CSF, consisting of prostatic acid phosphatase (PAP), an antigen expressed in prostate cancer tissue, linked to granulocyte-macrophage colony-stimulating factor (GM-CSF), an immune cell activator. Each dose of sipuleucel-T contains a minimum of 50 million autologous CD54+ cells activated with PAP-GM-CSF, suspended in 250 mL of Lactated Ringer's Injection, USP.

The active components of sipuleucel-T are autologous APCs and PAP-GM-CSF. During culture, the recombinant antigen binds to and is processed by APCs into smaller protein fragments. The recombinant antigen is designed to target APCs and may help direct the immune response to PAP. Minimal residual levels of the intact PAP-GM-CSF are detectable in the final sipuleucel-T product.

The patient's PBMCs are obtained via a standard leukapheresis procedure approximately 3 days prior to the infusion date. Due to the autologous nature of sipuleucel-T, it is important that the patient and physician adhere to the personalized leukapheresis and infusion schedules. The cellular composition of sipuleucel-T is dependent on the composition of cells obtained from the patient's leukapheresis. In addition to APCs, the final product contains T cells, B cells, natural killer (NK) cells, and other cells. The number of cells present and the cellular composition of each sipuleucel-T dose will vary.

The potency of sipuleucel-T is in part determined by measuring the increased expression of the CD54 molecule, also known as ICAM-1, on the surface of APCs after culture with PAP-GM-CSF. CD54 is a cell surface molecule that plays a role in the immunologic interactions between APCs and T cells and is considered a marker of immune cell activation. In this context, it is especially contemplated that N-803 will particularly enhance activation and proliferation of T cells, B cells, natural killer (NK) cells, and/or other immune cells. While not wishing to be bound by any theory or hypothesis, it is contemplated that N-803 will also beneficially increase antigen processing and presentation in antigen presenting cells (e.g., in dendritic cells and/or macrophages).

N-803: Both intravenous (IV) and subcutaneous (SC) dosing have been studied previously, with SC dosing showing better tolerability and pharmacokinetics (PK). Dose concentrations have ranged up to 20 micrograms/kilogram (mcg/kg). Another study (QuEST1 trial; NCT03493945) in mCRPC patients is looking at every 2 week dosing of N-803 up to 15 mcg/kg in combination with combination of a brachyury-targeting vaccine (BN-brachyury) and a TGF-β/anti-PD-L1 blocking bifunctional fusion protein (bintrafusp alfa). Thus, given the safety/tolerability to date both alone and in combination, the inventor contemplates using a starting dose of 20 mcg/kg SC, given every two weeks, concurrently with sip-T. As mentioned above, after the RP2D is determined in Cohort 1, enrollment continues first to Cohort 2 and then to Cohort 3. Both Cohorts 2 and 3 will have an additional dose of single agent N-803 (SC, at RP2D) administered either 1 week prior to the first sip-T+N-803 combination dosing, or 1 week after completion of the third combination dose. Such leading and trailing dosages is contemplated to lead to greater expansion/activation of sip-T cells, which may result in improved anti-tumor efficacy.

Metrics: With the proposed combination of Sip-T and N-803 in patients with mCRPC, it is anticipated that an improvement in clinical endpoints (e.g., PSA response, OS, PFS, etc) will be achieved as compared to Sip-T alone. For establishing safety and tolerability of the Sip-T and N-803 combination in patients with mCRPC, the frequency of dose-limiting toxicities by CTCAE v5.0 is used. Based on these results recommended phase II doses (RP2D) are established. Clinical and efficacy responses to the Sip-T and N-803 combination will be measured by PSA response (PSA30, PSA50, PSA90), radiographic progression free survival (PFS), failure-free survival (FFS, duration of time from date of treatment start to date of any progression events (radiographic progression, biochemical recurrence, death, whichever earlier)), time to next therapy (TTNT), overall survival (OS), and toxicity and severe adverse events per CTCAE v 5.0. Immune responses will be evaluated by quantitating peripheral immune responses (ELISPOT, Cytokines, CyTOF, humoral, T cell proliferation), tumor immune responses (pre- and post-treatment tissue), and PET imaging for tumor and immune response assessment.

In some embodiments, the numbers expressing quantities of ingredients, properties such as concentration, reaction conditions, and so forth, used to describe and claim certain embodiments of the invention are to be understood as being modified in some instances by the term “about.” As used herein, the terms “about” and “approximately”, when referring to a specified, measurable value (such as a parameter, an amount, a temporal duration, and the like), is meant to encompass the specified value and variations of and from the specified value, such as variations of +/−10% or less, alternatively +/−5% or less, alternatively +/−1% or less, alternatively +/−0.1% or less of and from the specified value, insofar as such variations are appropriate to perform in the disclosed embodiments. Thus, the value to which the modifier “about” or “approximately” refers is itself also specifically disclosed. The recitation of ranges of values herein is merely intended to serve as a shorthand method of referring individually to each separate value falling within the range. Unless otherwise indicated herein, each individual value is incorporated into the specification as if it were individually recited herein.

As used herein, the term “administering” a pharmaceutical composition or drug refers to both direct and indirect administration of the pharmaceutical composition or drug, wherein direct administration of the pharmaceutical composition or drug is typically performed by a health care professional (e.g., physician, nurse, etc.), and wherein indirect administration includes a step of providing or making available the pharmaceutical composition or drug to the health care professional for direct administration (e.g., via injection, infusion, oral delivery, topical delivery, etc.). It should further be noted that the terms “prognosing” or “predicting” a condition, a susceptibility for development of a disease, or a response to an intended treatment is meant to cover the act of predicting or the prediction (but not treatment or diagnosis of) the condition, susceptibility and/or response, including the rate of progression, improvement, and/or duration of the condition in a subject.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise. As also used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously.

It should be apparent to those skilled in the art that many more modifications besides those already described are possible without departing from the inventive concepts herein. The inventive subject matter, therefore, is not to be restricted except in the scope of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification or claims refer to at least one of something selected from the group consisting of A, B, C . . . and N, the text should be interpreted as requiring only one element from the group, not A plus N, or B plus N, etc.

What is claimed is:

1. A pharmaceutical composition comprising:
apheresis-derived peripheral blood mononuclear cells (PBMCs);
IL15:IL15RαSu or an agonist derivative thereof; and
a protein antigen.

2. The pharmaceutical composition of claim 1, wherein the protein antigen is a fusion protein.

3. The pharmaceutical composition of claim 2, wherein the fusion protein comprises prostatic acid phosphatase (PAP) and granulocyte-macrophage colony stimulating factor (GM-CSF).

4. The pharmaceutical composition of claim 3, wherein the fusion protein is PA2024.

5. The pharmaceutical composition of claim 1, wherein the agonist derivative of IL15:IL15RαSu is nogapendekin alpha inbakicept (N-803).

6. The composition of claim 1, wherein the PBMCs are from a human subject.

7. The composition of claim 1, wherein the PBMCs are enriched for monocytes, T cells, or natural killer (NK) cells.

8. The composition of claim 1, wherein the PBMC are treated with N-803 and PA2024, thereby stimulating the loading and MHC presentation of PAP by antigen presenting cells.

9. A pharmaceutical composition comprising:
IL15:IL15RαSu or an agonist derivative thereof; and
Sipuleucel-T (Sip-T).

10. The pharmaceutical composition of claim 9, wherein the agonist derivative of IL15:IL15RαSu is nogapendekin alpha inbakicept (N-803).

11. A method of treating a patient having metastatic castration-resistant prostate cancer (mCRPC), the method comprising the steps of:
obtaining peripheral blood mononuclear cells (PBMCs) from the patient;
activating ex vivo the PBMCs with IL15:IL15RαSu or an agonist derivative thereof and a prostate cancer antigen to provide an enhanced immunotherapy sipuleucel-T (Sip-T); and
administering the enhanced immunotherapy Sip-T to the patient.

12. The method of claim 11, wherein the prostate cancer antigen is a fusion protein.

13. The method of claim 12, wherein the fusion protein comprises prostatic acid phosphatase (PAP) and granulocyte-macrophage colony stimulating factor (GM-CSF).

14. The method of claim 13, wherein the fusion protein is PA2024.

15. The method of claim 11, wherein the agonist derivative of IL15:IL15RαSu is nogapendekin alpha inbakicept (N-803).

16. The method of claim 11, wherein the PBMCs are from the patient.

17. The method of claim 11, wherein the PBMCs are enriched for monocytes, T cells, and/or natural killer (NK) cells.

18. The method of claim 11, wherein the PBMC are treated with N-803 and PA2024, thereby stimulating the loading and MHC presentation of PAP by antigen presenting cells.

19. The method of claim 11, further comprising a step of administering N-803 the patient before administering the enhanced immunotherapy Sip-T to the patient.

20. The method of claim 11, further comprising a step of administering N-803 the patient after administering the enhanced immunotherapy Sip-T to the patient.
